(19) United States
(12) Patent Application Publication
(10) Pub. No.: US 2026/0263001 A1
(43) Pub. Date: Sep. 10, 2026

(54) NONINVASIVE BLOOD ALCOHOL CONCENTATION (BAC) MEASUREMENT SYSTEM AND METHOD THEREOF

(71) Applicant: Vivonics, Inc., Bedford, MA (US)

(72) Inventors: Michele Pierro, Westford, MA (US); Gordon B. Hirschman, Cohoes, NY (US)

(21) Appl. No.: 19/553,694

(22) Filed: Mar. 2, 2026

Related U.S. Application Data
(60) Provisional application No. 63/766,460, filed on Mar. 4, 2025.

(51) Int. Cl. A61B 5/00; A61B 5/1455

(52) U.S. Cl. CPC A61B 5/4845; A61B 5/1455

(57) ABSTRACT

A noninvasive BAC measurement system includes one or more emitters configured to emit light at one or more wavelengths sensitive to concentrations of alcohol in blood of a human subject and one or more emitter configured to emit light at one or more wavelengths less sensitive to concentrations of alcohol in the blood of the human subject. One or more detectors are configured to detect light emitted at the one or more wavelengths sensitive to concentrations of alcohol and detect light emitted at the one or more wavelengths less sensitive to concentrations of alcohol and generate a waveform for each of the one or more wavelengths sensitive to concentrations of alcohol and a waveform for each of one or more wavelengths less sensitive to concentrations of the alcohol. A processing subsystem compares at least one waveform for each of the one or more wavelengths sensitive to concentrations of alcohol and at least one waveform for each of the one or more wavelengths less sensitive to concentrations of alcohol to determine the BAC.

### RELATED APPLICATIONS

This application claims benefit of and priority to U.S. Provisional Application Ser. No. 63/766,460 filed Mar. 4, 2025 under 35 U.S.C. §§ 119, 120, 363, 365, and 37 C.F.R. § 1.55 and § 1.78, incorporated by reference herein.

### GOVERNMENT RIGHTS

This invention was made with U.S. Government support under Contract No. 75N94023C00012 awarded by the National Institutes of Health. The Government has certain rights in the invention.

### FIELD OF THE INVENTION

This invention relates to a noninvasive blood alcohol concentration (BAC) measurement system and method thereof.

### BACKGROUND OF THE INVENTION

Alcohol abuse is an ever-present problem throughout human history and societies and may be responsible for about 20 deaths worldwide. In the United States alone, alcohol use disorders may affect almost 15 million people and have an economic cost of about $250 billion. The great importance and tremendous impact that alcohol abuse has had on our society and economy has led researchers to understand the psychological impacts, health implications, and social ramifications of alcohol consumption, as well as the best ways to treat alcohol abuse disorders.

The social consumption of alcohol is a normalized means of social gathering for many Americans and countries around the world, with business deals, commerce, weeklong events, and celebrations tied in one way or another to the act for a significant number of cultures. At the same time, the abuse of alcohol has long plagued civilizations, seemingly regardless of their cultural use, driving researchers to understand the psychological impacts, health implications, and social ramifications of alcohol consumption. Due to the stigma towards alcohol abuse in the United States, the raw data to support this research has been historically difficult to come by, relying heavily on short term studies and patient questionnaires that rely on human nature for accuracy. There has been a great deal of research around alcohol and the benefits and detriments to consuming it. BAC has long been linked to a reduction in driver safety and competency, leading to a higher frequency of automobile accidents as BAC increases. Studies have even shown that the slightest bit of alcohol in a driver's system (0.01%) can significantly increase the severity of automobile accidents.

Thus, there is a need for a system and method which can noninvasively, accurately, and effectively measure BAC.

### SUMMARY OF THE INVENTION

In one aspect, a noninvasive blood alcohol concentration (BAC) measurement system is featured. The system includes one or more emitters configured to emit light at one or more wavelengths sensitive to concentrations of alcohol in blood of a human subject and one or more emitters configured to emit light at one or more wavelengths less sensitive to concentrations of alcohol in the blood of the human subject. One or more detectors are configured to detect light emitted at the one or more wavelengths sensitive to concentrations of alcohol and detect light emitted at the one or more wavelengths less sensitive to concentrations of alcohol and generate a waveform for each of the one or more wavelengths sensitive to concentrations of alcohol and a waveform for each of one or more wavelengths less sensitive to concentrations of the alcohol. A processing subsystem is coupled to the one or more emitters and the one or more detectors and is configured to compare at least one waveform for each of the one or more wavelengths sensitive to concentrations of alcohol and at least one waveform for each of the one or more wavelengths less sensitive to concentrations of alcohol to determine the BAC.

In one example, the processing subsystem may determine a sensitive metric of power in each waveform of the one or more wavelengths sensitive to concentrations of alcohol and a less sensitive metric of power for each waveform of the one or more wavelengths less sensitive to concentrations of the alcohol and determines BAC by comparing the sensitive metric of power to the less sensitive metric of power. The sensitive metric of power and the less the sensitive metric of power may include at least one of: an area under the waveform of the one or more wavelengths sensitive to concentrations of alcohol, an area under the waveform of the one or more wavelengths less sensitive to concentrations of alcohol, a square root of the area under the waveform of the one or more wavelengths sensitive to concentrations of alcohol, a square root of the area under the waveform of the one or more wavelengths less sensitive to concentrations of alcohol, a sum of the squares of the values in the waveform of the one or more wavelengths sensitive to concentrations of alcohol, a sum of the squares of the values in the waveform of the one or more wavelengths less sensitive to concentrations of alcohol, a root mean square of the values in the waveform of the one or more wavelengths sensitive to concentrations of alcohol, a root mean square of the values in the waveform of the one or more wavelengths sensitive to concentrations of alcohol, a metric of peak power, a metric of average power, or a metric of weighted average power. The processing subsystem may be configured to perform frequency domain analysis of each waveform of the one or more wavelengths sensitive to concentrations of alcohol and produce a sensitive power spectral density curve corresponding to hemodynamic oscillations over a predetermined frequency range, perform frequency domain analysis of each waveform of the one or more wavelengths less sensitive to concentrations of alcohol and produces a less sensitive power spectral density curve corresponding to hemodynamic oscillations over the predetermined frequency range, calculate a metric of power in the sensitive power spectral density curve, calculate a metric of power in the less sensitive power spectral density curve, and determine the BAC by comparing the metric of power in the sensitive power spectral density curve to the metric of power in the less sensitive power spectral density curve. The processing subsystem may be configured to determine BAC by calculating an area under the sensitive power spectral density curve, calculating an area under the less sensitive power spectral density curve, and determining a ratio of the calculated area under the sensitive power spectral density curve to the calculated area under the area under the less sensitive power spectral density curve. The predetermined frequency range may be in the range of about 0.5 to about 3 Hz. The one or more wavelengths sensitive to concentrations of alcohol may be in the range of about 1500 nm to about 2400 nm. The one or more wavelengths less sensitive to concentrations of alcohol may be in the range of about 1,200 nm to about 1,900 nm. The processing subsystem may be configured to rapidly and sequentially turn on and turn off one or more of the one or more emitters emitting light at the one or more wavelengths sensitive to concentrations of alcohol and emitting light the one or more wavelengths less sensitive to concentrations of alcohol in the blood of the human subject to multiplex the signal to the one or more detectors. The one or more emitters and the one or more detectors may be adapted to be placed against the skin of a portion of a human subject adjacent to each other. The one or more emitters may be adapted to be placed against the skin on a portion of a human subject and the one or more detectors may be adapted to be placed against the skin of a human subject on the opposite side of the portion of the human subject.

In another example, a noninvasive blood alcohol concentration (BAC) measurement method is featured. The method includes emitting light at one or more wavelengths sensitive to concentrations of alcohol in blood of a human subject, emitting light at one or more wavelengths less sensitive to concentrations of alcohol in the blood the human subject, detecting the light emitted at the one or more wavelengths sensitive to concentrations of alcohol, and detecting the light emitted at one or more wavelengths less sensitive to concentrations of alcohol. The method also includes generating a time-varying waveform for each of the one or more wavelengths sensitive to concentrations of alcohol and a time-varying waveform for each of one or more wavelengths less sensitive to concentrations of the alcohol, and comparing at least one waveform for each of the one or more wavelengths sensitive to concentrations of alcohol and at least one waveform for each of the one or more wavelengths less sensitive to concentrations of alcohol to determine the BAC.

In one example, the method may include determining a sensitive metric of power in each waveform of the one or more wavelengths sensitive to concentrations of alcohol and determining a less sensitive metric of power for each waveform of the one or more wavelengths less sensitive to concentrations of the alcohol. The sensitive metric of power and the less the sensitive metric of power include at least one of: an area under the waveform of the one or more wavelengths sensitive concentrations of alcohol, an area under the waveform of the one or more wavelengths less sensitive concentrations of alcohol, a square root of the area under the waveform of the one or more wavelengths sensitive to concentrations of alcohol, a square root of the area under the waveform of the one or more wavelengths less sensitive to concentrations of alcohol, a sum of the squares of the values in the waveform of the one or more wavelengths sensitive to concentrations of alcohol, a sum of the squares of the values in the waveform of the one or more wavelengths less sensitive to concentrations of alcohol, a root mean square of the values in the waveform of the one or more wavelengths sensitive to concentrations of alcohol, a root mean square of the values in the waveform of the one or more wavelengths sensitive to concentrations of alcohol, a metric of peak power, a metric of average power, or a metric of weighted average power. The method may include determining BAC by comparing the sensitive metric of power to the less sensitive metric of power. The method may include performing frequency domain analysis of each time-varying waveform of the one or more wavelengths sensitive to concentrations of alcohol and producing a sensitive power spectral density curve corresponding to hemodynamic oscillations over a predetermined frequency range, performing frequency domain analysis of each waveform of the one or more wavelengths less sensitive to concentrations of alcohol and produces a less sensitive power spectral density curve corresponding to hemodynamic oscillations over the predetermined frequency range, calculating a metric of power in the sensitive power spectral density curve, calculating a metric of power in the less sensitive power spectral density curve, and determining the BAC by comparing the metric of power in the sensitive power spectral density curve to the metric of power in the less sensitive power spectral density curve. The BAC may be determined by calculating an area under the sensitive power spectral density curve, calculating an area under the less sensitive power spectral density curve, and determining a ratio of the calculated area under the sensitive power spectral density curve to the calculated area under the area under the less sensitive power spectral density curve. The predetermined frequency range may be in the range of about 0.5 to about 3 Hz. The one or more wavelengths sensitive to concentrations of alcohol may be in the range of about 1500 nm to about 2400 nm. The one or more wavelengths less sensitive to concentrations of alcohol may be in the range of about 1,200 nm to about 1,900 nm. The method may include rapidly and sequentially emitting light at the one or more wavelengths sensitive to concentrations of alcohol and rapidly and sequentially emitting light the one or more wavelengths less sensitive to concentrations of alcohol. The method may include emitting only light in one narrow frequency range sensitive to concentrations of alcohol in the blood and/or emitting only light in one narrow frequency range less sensitive to concentrations of alcohol.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic block diagram showing the primary components of one example of the noninvasive blood alcohol concentration (BAC) measurement system;

FIG. 2 shows examples of different wavelengths sensitive to BAC and different wavelengths less sensitive to BAC;

FIG. 3 shows an example of the system shown in FIG. 1 generating and comparing time-varying waveforms sensitive and less sensitive to concentrations of alcohol and then comparing those waveforms to determine BAC;

FIG. 4 is a three-dimensional view of an example of the system shown in one or more of FIGS. 1-3 integrated as patch;

FIG. 5A shows an example of the patch shown in FIG. 4 in place on a human subject;

FIG. 5B shows in further detail the structure of the patch shown in FIGS. 4A and 5A;

FIG. 6 is a schematic block diagram showing an example of the system shown in one or more of FIGS. 1-5B operating in transmission mode; and

FIG. 7 is a flow chart showing the primary steps of one example of the noninvasive blood alcohol concentration (BAC) measurement method.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

The noninvasive blood alcohol concentration (BAC) measurement system and method preferably quantifies alcohol concentration in blood by measuring and processing data at selected wavelengths within the near infrared spectrum in a manner that is preferably easily integrable into a patch-like form factor or may be integrated into a wrist strap, a wrist watch, a ring, a piece of jewelry, a garment (e.g., shirt, pants, hat, glove, and the like), eyeglasses, an earpiece, a headphone, a locked ankle bracelet, or similar type device.

One goal of the noninvasive blood alcohol concentration (BAC) measurement system and method is preferably to advance scientific knowledge about the nature of alcohol use disorders and reduce the impact of alcohol intoxication on driving fatalities and other public health problems. Despite recent advances in the biomedical industry, which has made significant strides in creating various technologies for measuring blood alcohol concentration, there is still a technology gap for continuous blood alcohol concentration monitoring in an accurate, precise, and discreet manner. This is especially true in a clinical setting, where the full impact of alcohol dosage and frequency on short term and long term physiological, psychological, and social wellness is understood only at a high level. Many existing conventional technologies, while seeing a great deal of use in law enforcement and private industry, have yet to be applied to treating alcohol abuse in a clinical setting or for pharmaceutical testing and evaluation purposes.

The noninvasive blood alcohol concentration (BAC) measurement system and method preferably allows users to collect high resolution and accurate quantitative data sets of a subject's BAC to fully understand how the frequency and dosage of alcohol in the body correlates to overall health, preferable in near real time for continuous monitoring. As will be discussed below, the noninvasive blood alcohol concentration (BAC) measurement system and method preferably has a low profile, contacts the body, and features sensors that can quantify alcohol concentration within a few minutes after measuring. The noninvasive blood alcohol concentration (BAC) measurement system and method preferably provides a positive impact on public health and quality of life. The noninvasive blood alcohol concentration (BAC) measurement system and method may be utilized as a consumer device, allowing individuals to monitor their alcohol intake in an accurate and immediate way.

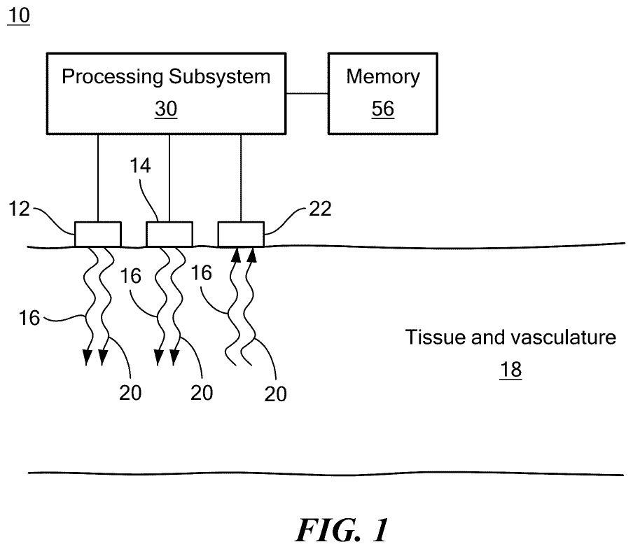

There is shown in FIG. 1, one example noninvasive blood alcohol concentration (BAC) measurement system 10 and method thereof. System 10 preferably includes one or more emitters, e.g., emitters 12 and 14, which emit light 16 at one or more wavelengths sensitive to concentrations of alcohol in the blood in tissue and vasculature 18 of a human subject. In one example, the one or more wavelengths sensitive to concentrations of alcohol in the blood in tissue and vasculature 18 are preferably in range of about 1500 nm to about 2400 nm. In this example, emitter 12 and/or 14 may emit one or two wavelengths sensitive to concentrations of alcohol in the blood of tissue and vasculature 18 or may emit three, four, five, or any desired number of wavelengths sensitive to concentrations of alcohol in the blood in tissue and vasculature 18.

One or more emitters 12, 14 also preferably emit light 20 at one or more wavelengths less sensitive to concentrations of alcohol in the blood in tissue and vasculature 18 of the human subject, e.g., wavelengths in the range of about 1,200 nm to about 1,900 nm. Similarly, emitter 12 and/or emitter 14 may emit one or two wavelengths less sensitive to concentrations of alcohol in the blood in tissue and vasculature 18 or may emit three, four, five, or any desired number of wavelengths less sensitive to concentrations of alcohol in blood in tissue and vasculature 18.

In one design, the one or more emitters 12, 14 are preferably light emitting diodes (LEDs). In other examples, the one or more emitters 12, 14 may be organic light emitting diodes (OLEDs), quantum dot LEDs, laser diodes, or similar type devices.

Although in the example discussed above, each of emitters, 12, 14 emit light 16 at one or more wavelengths sensitive to concentrations of alcohol in the blood in tissue and vasculature 18 and emit light 20 at one or more wavelengths less sensitive to concentrations of alcohol in the blood in tissue and vasculature 18, in other examples, one emitter may emit light 16 at one or more wavelengths sensitive to concentrations of alcohol in the blood in tissue and vasculature 18 and another emitter may emit light 20 at one or more wavelengths less sensitive to concentrations of alcohol in the blood in tissue and vasculature 18. In other examples, there may be three, four, five, or more emitters which each may emit light 16 or emit light 20, as needed and known by one skilled in the art.

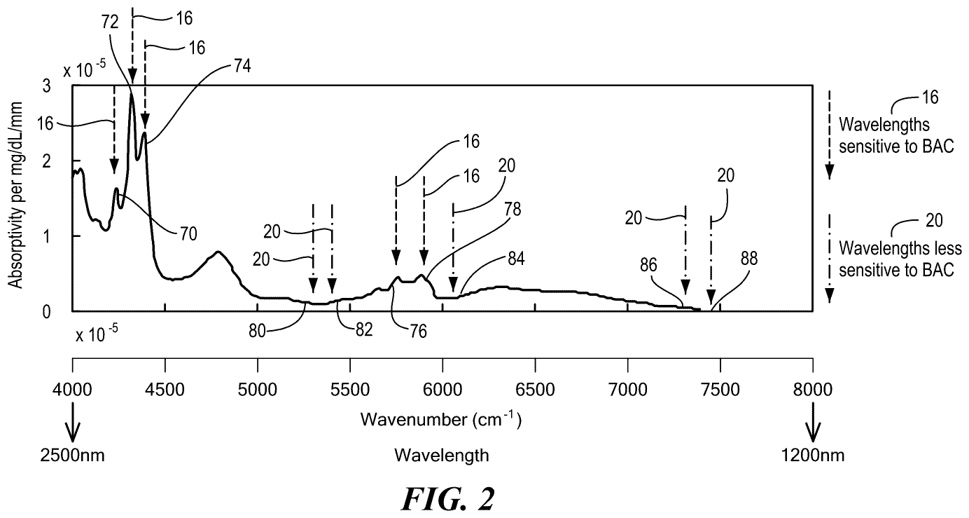

FIG. 2, where like parts have like numbers, shows in further detail an example where the one or more emitters of system 10 emit light 16 at different wavelengths sensitive to BAC, e.g., as indicated at 70, 72, 74, 76, and 78 and emit light 20 at different wavelengths less sensitive to BAC as shown, e.g., as indicated at 80, 82, 84, 86, and 88.

System 10 also preferably includes one or more detectors, e.g., detector 22, FIG. 1, which detects reflected light 16 emitted at the one or more wavelengths sensitive to concentrations of alcohol and detects reflected light 20 emitted at one or more wavelengths less sensitive to concentrations of alcohol and generates a time-varying waveform for each of the one or more wavelengths sensitive to concentrations of alcohol and a time-varying waveform for each of one or more wavelengths less sensitive to concentrations of the alcohol.

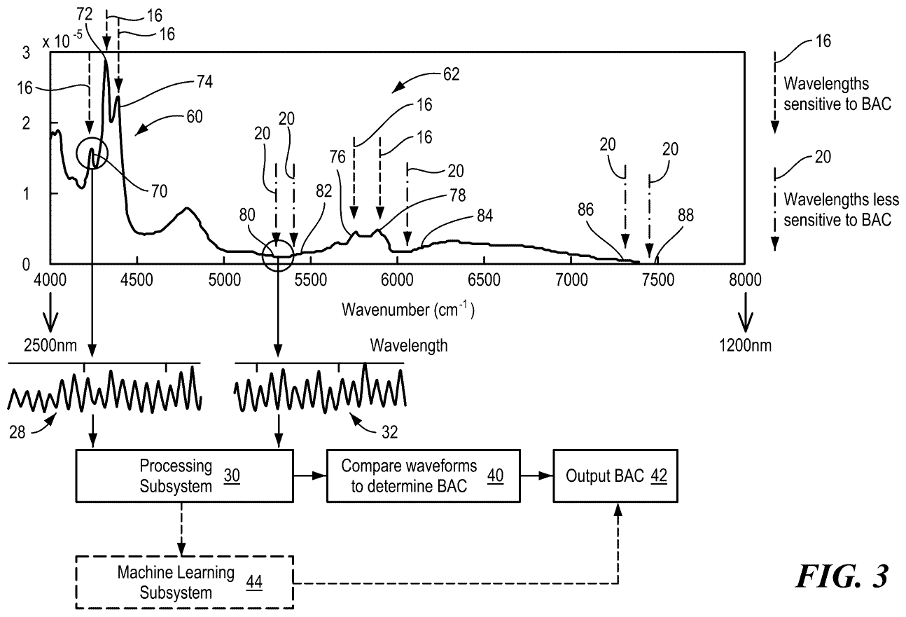

FIG. 3, where like parts have like numbers, shows an example in which detector 22, FIG. 1, detects light 16 at the one or more wavelengths sensitive to concentrations of alcohol, e.g., indicated at 70, FIG. 3 and generates time-varying waveform 28. Similarly, in this example, detector 22 detects light 20 at the one or more wavelengths less sensitive to concentrations of alcohol, e.g., indicated at 80, and generates time-varying waveform 32. As disclosed herein, sensitive means that the presence of alcohol will impact the amount of light that will reach detector 22. For example, more alcohol in the blood in tissue and vasculature 18 will lead to more absorption so less light will reach detector 22. In this non-limiting example, system 10 may utilize five wavelengths sensitive to BAC, e.g., wavelengths 70, 72, 74, 76, and 78, and five wavelengths less sensitive to BAC, e.g., wavelengths 80, 82, 84, 86, and 88. In other examples, system 10 may utilize more or less than five wavelengths sensitive to BAC and may utilize more or less than five wavelengths less sensitive to BAC, as needed and known by those skilled in the art. The number wavelengths sensitive to BAC and the number of wavelengths less sensitive to BAC do not have to be the same.

System 10, FIG. 1, also preferably includes processing subsystem 30 coupled to the one or more emitters 12, 14 and the one or more detectors 22. In one example, processing subsystem 30, also shown in FIG. 3, preferably compares waveforms, indicted at 40, to determine BAC, indicated at 42. For example, system 10 may compare at least one waveform for each of the one or more wavelengths sensitive to concentrations of alcohol, e.g., waveform 28 for wavelength 70 of light 16, and at least one waveform for each of the one or more wavelengths less sensitive to concentrations of alcohol, e.g., waveform 32 for wavelength 80 of light 20, to determine the BAC. In other examples, processing subsystem 30 may compare other waveforms for different wavelengths sensitive to concentrations of alcohol, e.g., any of wavelengths 72, 74, 76 and/or 78 to other waveform for different wavelengths less sensitive to concentrations of alcohol, e.g., any of wavelengths 82, 84, 86, and/or 88.

In one embodiment, processing subsystem 30 may determine a sensitive metric of power in each waveform of the one or more wavelengths sensitive to concentrations of alcohol, e.g. waveform 28 at wavelength 70, and determine a less sensitive metric of power for each waveform of the one or more wavelengths less sensitive to concentrations of the alcohol, e.g., waveform 32 at wavelength 80 and determine BAC by comparing the sensitive metric of power and the less sensitive metric of power.

In one example, the sensitive metric of power includes at least one of: an area under the waveform of the one or more wavelengths sensitive to concentrations of alcohol, a square root of the area under the waveform of the one or more wavelengths sensitive to concentrations of alcohol, a sum of the squares of the values in the waveform of the one or more wavelengths sensitive to concentrations of alcohol, a root mean square of the values in the waveform of the one or more wavelengths sensitive to concentrations of alcohol, a metric of peak power, a metric of average power, or a metric of weighted average power.

Similarly, the less sensitive metric of power may include at least one of: an area under the waveform of the one or more wavelengths less sensitive to concentrations of alcohol, a square root of the area under the waveform of the one or more wavelengths less sensitive to concentrations of alcohol, a sum of the squares of the values in the waveform of the one or more wavelengths less sensitive to concentrations of alcohol, a root mean square of the values in the waveform of the one or more wavelengths less sensitive to concentrations of alcohol, a metric of peak power, a metric of average power, or a metric of weighted average power.

In another example, processing subsystem 30 may perform frequency domain analysis of each time-varying waveform of the one or more wavelengths sensitive to concentrations of alcohol and produces a sensitive power spectral density curve corresponding to hemodynamic oscillations over a predetermined frequency range. Similarly, processing subsystem 30 also preferably performs frequency domain analysis of each time-varying waveform of the one or more wavelengths less sensitive to concentrations of alcohol and produces a less sensitive power spectral density curve corresponding to hemodynamic oscillations over the predetermined frequency range.

In another example, processing subsystem 30, FIGS. 1 and 3, may perform frequency domain analysis of waveform 28 at wavelength 70 which is preferably sensitive to concentrations of alcohol and produce a sensitive power spectral density curve corresponding to hemodynamic oscillations over a predetermined frequency range, e.g., a frequency in the range of about 0.5 to about 3 Hz. Similarly, in this example, processing subsystem 30, may perform frequency domain analysis of waveform 32, at wavelength 80 which is preferably less sensitive to concentrations of alcohol and produces less sensitive power spectral density curve corresponding to hemodynamic oscillations over a predetermined frequency range, e.g., a frequency in the range of about 0.5 to about 3 Hz.

Processing subsystem 30 may also calculate a sensitive metric of power from the sensitive power spectral density curve for sensitive power spectral density curve and may calculate a less sensitive metric of power from the less sensitive power spectral density curve for less sensitive power spectral density curve and determines the BAC by comparing the metric of power from the sensitive power spectral density curve to the metric of power from the less sensitive power spectral density curve.

In one design, the metric of power in the sensitive power spectral density curve may include the area under the sensitive power spectral density curve. Similarly, the metric of power in the less sensitive power spectral density curve may include the area under the less sensitive power spectral density curve. In this example, processing subsystem 30 may determine BAC by comparing the area under the sensitive power spectral density curve to the area under less sensitive power spectral density curve. In this example, the ratio between the areas may be utilized to determine BAC.

In another example, processing subsystem 30, FIG. 3, may utilize machine learning subsystem 44 to compare waveforms 28 and waveforms 32 to determine BAC, indicated at 42.

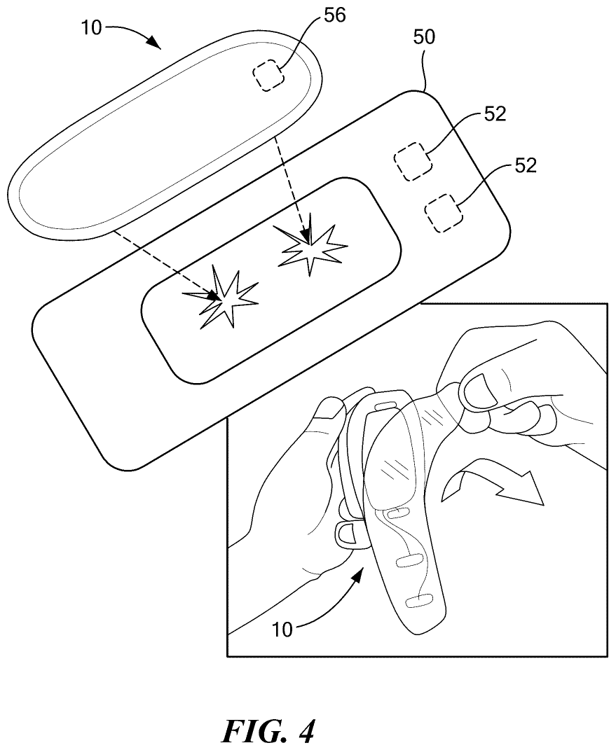

As discussed above, system 10 may be integrable into a patch like form factor e.g., as shown in FIG. 4, with snap-in-place board 50 which preferably includes one or more optical-based sensors, e.g., the one or more emitters and one or more detectors discussed above, and preferably includes processor system 30. Preferably board 50 with the sensors is reusable and can be washed and disinfected multiple times. The power supply for system 10, FIG. 1, e.g., battery 52, FIG. 4, may be built into or integrated with reusable board 50 and can preferably be recharged by removing the sensor board from the patch and charging it through a mini-USB port. In an alternate embodiment, board 50 may be permanently integrated into a disposable patch like form factor implementation of system 10.

As also discussed above, system 10 may be integrated into a wrist strap, or wrist watch, a ring, a piece of jewelry, a garment (e.g., shirt, pants, hat, glove, and the like), eyeglasses, an earpiece, a headphone, a locked ankle bracelet, or similar type device, having similar features to the embodiment discussed above with reference to FIG. 4.

In one prototype design, system 10 preferably includes about two to about ten emitters emitting light 16 and/or light 20 at about two to ten different wavelengths and about one multiplexed photodetector for each pair (2) of emitters, comprised of one sensitive wavelength and one less sensitive wavelength.

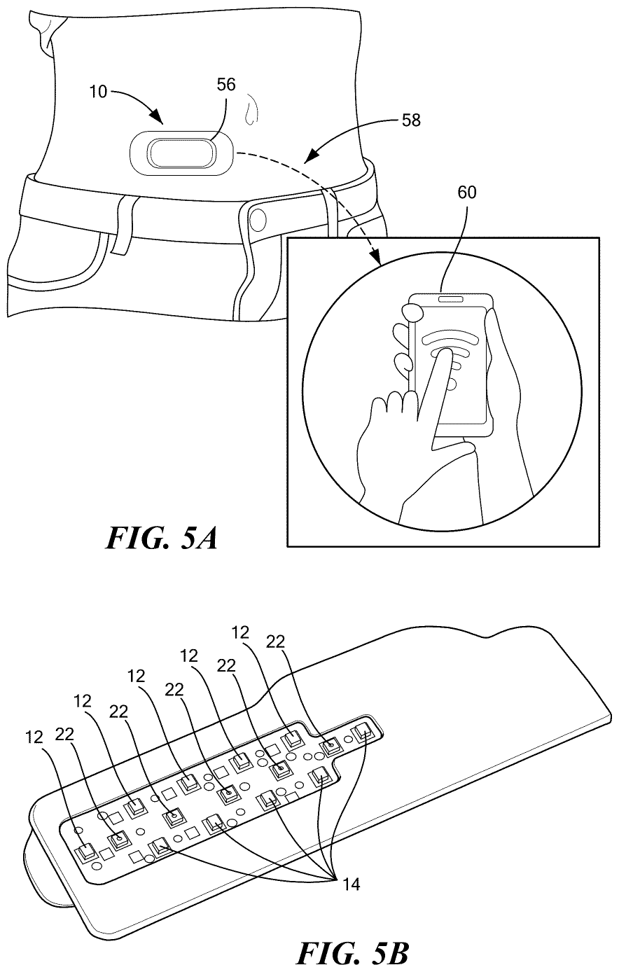

System 10 and method thereof for noninvasively measuring BAC using light preferably detects and quantifies blood alcohol levels by leveraging optical spectroscopy and light diffusion and propagation in tissue. This technology preferably relies on the basic principles of light diffusion and propagation in tissue by measuring changes in absorbance directly from the subject's blood and tissue through reflected light at specific selected wavelengths that maximize the sensitivity to alcohol concentration in blood. See e.g., Lorenzo, J. R., Principles of diffuse light propagation: light propagation in tissues with applications in biology and medicine, World Scientific (2012); Tuchin, V., et al., Light propagation in tissues with controlled optical properties in photon propagation in tissues II, SPIE (1996.); Ridder, T. e. al., Noninvasive alcohol testing using diffuse reflectance near-infrared spectroscopy, Applied Spectroscopy, 59(2): p. 181-189 (2005); and Ridder, T. et al., Comparison of spectroscopically measured tissue alcohol concentration to blood and breath alcohol measurements, Journal of biomedical optics, 14(5), p. 054039 (2009), all incorporated by reference herein. The reflected and collected light carries real-time information about alcohol concentration changes in blood, information that is preferably stored locally in memory 56, FIGS. 1 and 4 of system 10, and may be wirelessly and intermittently transmitted, e.g., indicated at 58, FIG. 5, e.g. using Bluetooth or similar type transmission, to smart device 60, e.g., a phone, tablet, personal computer or similar type device.

As discussed above with reference to one or more of FIGS. 1-5B, system 10 and the method thereof for noninvasively measuring BAC using light may operate in reflectance mode, where system 10 measures changes in absorbance from the subject's blood and tissue through light that passes through these layers and scatters or reflects back toward the direction from which it came, and preferably includes an array consisting of a plurality of light emitters (LEDs) and a plurality of photodetectors located on the same side of the tissue and vasculature as discussed above to capture data and non-invasively measure BAC. One example of the design of such a system fabricated in a patch-like format is shown in FIG. 5B, where like parts have been giving numbers.

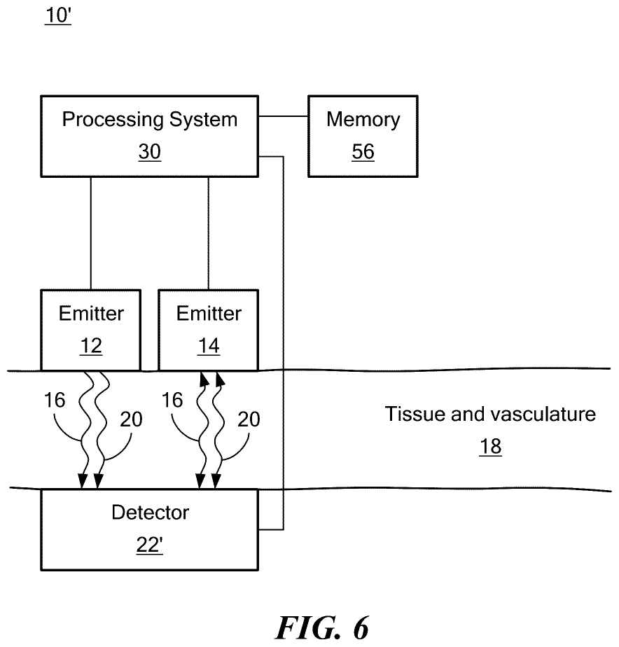

In another example, noninvasive BAC measurement system 10′, FIG. 6, where like parts have like numbers, system 10′ and the method thereof may operate in transmission mode. In this example, one or more emitters 12, 14 preferably emit light 16 at one or more wavelengths sensitive to concentrations of alcohol in the blood of tissue and vasculature 18 and preferably emit light 20 at one or more wavelengths less sensitive to concentrations of alcohol in the blood of tissue and vasculature 18, similar as discussed above with reference to one or more of FIGS. 1-5.

However, in this example, system 10′, FIG. 6, preferably includes one or more detectors adapted to be located on the opposite side of a portion of the human subject from the emitters, e.g., detector 22′, which detects light 16 emitted at the one or more wavelengths sensitive to concentrations of alcohol and detects light 20 emitted at one or more wavelengths less sensitive to concentrations of alcohol that have been transmitted through tissue and vasculature 18 as shown. Detector 22′ then preferably generates a waveform for each of the one or more wavelengths sensitive to concentrations of alcohol and a waveform for each of one or more wavelengths less sensitive to concentrations of the alcohol, similar as discussed above. Preferably, the one or more emitters 12, 14 and the one or more detectors, e.g., detector 22′, are adapted to be placed on opposite sides of a portion of the human subject, such as a finger, a wrist, an ear, or other relatively thin portion of the human subject, where the optical path between emitters and detectors is relatively short to avoid excessive loss of optical power at the detector.

Preferably, system 10 or 10′ and the method thereof preferably measures spontaneous hemodynamic oscillations at predetermined frequencies, e.g., in the range of about 0.5 to about 3 Hz, and at the wavelengths discussed above that are preferably selected to detect alcohol concentration in blood to determine BAC. System 10 and the method thereof is preferably resilient to motion artifacts, ambient light, and other confounding factors such as water and other chromophores present in blood and tissues. Among these chromophores, melanin can contribute to the signal by adding DC components, masking the effect of alcohol concentration on absorbance changes in the time-domain. In one design, the power level of the incident light may be adjusted, preferably depending on the power level of the detected light, in order to adjust for varying levels of melanin in the human subject. In one example, an initial calibration of system 10 may be performed by modulating the power to the one or more emitters 12, 14, e.g., LEDs, to an optimal point based on the signal levels detected by the one or more detectors 22, as known by those skilled in the art.

Absorbance changes and respective blood volume oscillations may preferably be extrapolated by using the modified Beer-Lambert Law. See e.g., Pierro, M. L., et al., Phase-amplitude investigation of spontaneous low-frequency oscillations of cerebral hemodynamics with near-infrared spectroscopy: a sleep study in human subjects, Neuroimage, 63(3): p. 1571-1584 (2012) and Sassaroli, A., et al., Low-frequency spontaneous oscillations of cerebral hemodynamics investigated with near-infrared spectroscopy: a review, IEEE Journal of Selected Topics in Quantum Electronics, 18(4): p. 1478-1492 (2012), both incorporated by reference herein. Frequency domain (FFT) analysis, can be used to detect components at the specific frequencies of oscillation, for example at the respiratory rate, and present across the multiple selected wavelengths, as discussed above.

Processing subsystem 30, FIGS. 1, 3, and 6, may be configured to rapidly and sequentially turn on and turn off one or more of the one or more emitters emitting light at the one or more wavelengths sensitive to concentrations of alcohol in the blood in tissue and vasculature 18 and emitting light the one or more wavelengths less sensitive to concentrations of alcohol in the blood the human subject.

In one example, the waveform generated from detector 22, 22′ preferably demultiplexes the signal by sampling at times synchronous with the times when each of the one or more emitters are rapidly and sequentially turned on and off, to generate a separate sampled waveform associated with each of the one or more emitters 12, 14. This enables system 10 to have fewer detectors than emitters. As is known to one skilled in the art, the sampling rate should be selected to be at least twice the frequency of the highest frequency component of interest in the spontaneous hemodynamic oscillations and preferably a higher multiple of the highest frequency component of interest in the spontaneous hemodynamic oscillations.

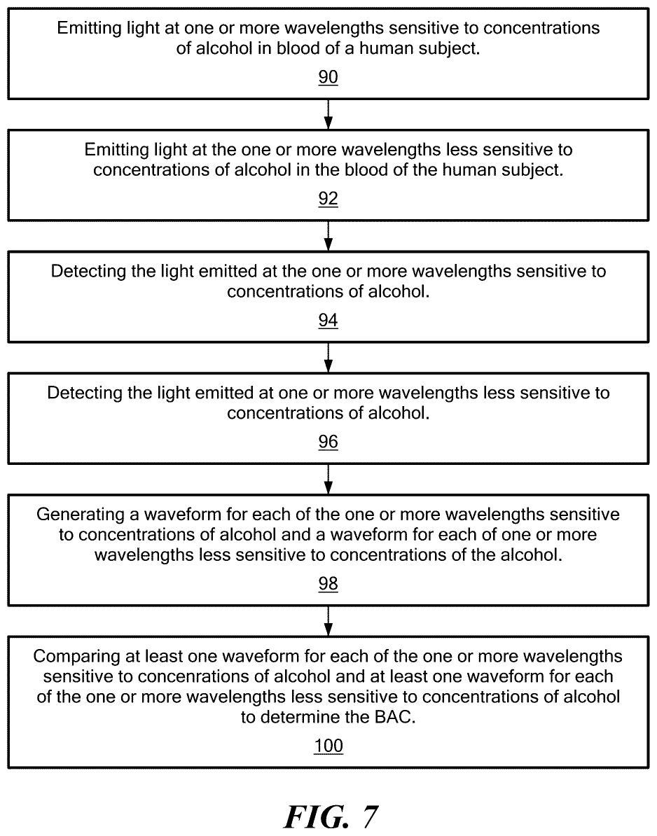

One example of the noninvasive blood alcohol concentration (BAC) measurement method preferably includes emitting light at one or more wavelengths sensitive to concentrations of alcohol in blood of a human subject, step 90, FIG. 7, emitting light at the one or more wavelengths less sensitive to concentrations of alcohol in the blood the human subject, step 92, detecting the light emitted at the one or more wavelengths sensitive to concentrations of alcohol, step 94, and detecting the light emitted at one or more wavelengths less sensitive to concentrations of alcohol, 96. The method also preferably includes generating a time varying waveform for each of the one or more wavelengths sensitive to concentrations of alcohol and a time-varying waveform for each of one or more wavelengths less sensitive to concentrations of the alcohol, step 98, and comparing at least one waveform for each of the one or more wavelengths sensitive to concentrations of alcohol and at least one waveform for each of the one or more wavelengths less sensitive to concentrations of alcohol to determine the BAC, step 100.

In one example, processing subsystem 30 may perform one or more operations or steps discussed above with reference to one or more of FIGS. 1-7 in real-time or near real-time, as known by those skilled in the art.

In one embodiment, each of the one or more emitters 12, 14 of system 10 or 10′ may exclusively emit only light in one narrow frequency range that is preferably sensitive to concentrations of alcohol in the blood in tissue and vasculature 18 and/or preferably less sensitive to concentrations of alcohol in the blood in tissue and vasculature 18. This narrow frequency range may be implemented through inherent design of the emitter device or achieved by adding an optical filter at the output of an emitter device that inherently emits multiple frequencies.

The result is system 10 and the method thereof non-invasively, efficiently, and effectively determines BAC. System 10 is preferably small, compact, low profile, and can easily be worn and concealed by the user if desired and can efficiently collect high resolution and accurate quantitative data for analysis and study by health care professionals. System 10 may be utilized as a consumer device, allowing individuals to monitor their alcohol intake in an accurate and immediate way.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A noninvasive blood alcohol concentration (BAC) measurement system, the system comprising:
one or more emitters configured to emit light at one or more wavelengths sensitive to concentrations of alcohol in blood of a human subject and one or more emitters configured to emit light at one or more wavelengths less sensitive to concentrations of alcohol in the blood of the human subject;
one or more detectors configured to detect light emitted at the one or more wavelengths sensitive to concentrations of alcohol and detect light emitted at the one or more wavelengths less sensitive to concentrations of alcohol and generate a waveform for each of the one or more wavelengths sensitive to concentrations of alcohol and a waveform for each of one or more wavelengths less sensitive to concentrations of the alcohol; and
a processing subsystem coupled to the one or more emitters and the one or more detectors configured to:
compare at least one waveform for each of the one or more wavelengths sensitive to concentrations of alcohol and at least one waveform for each of the one or more wavelengths less sensitive to concentrations of alcohol to determine the BAC.

2. The system of claim 1 in which the processing subsystem determines a sensitive metric of power in each waveform of the one or more wavelengths sensitive to concentrations of alcohol and a less sensitive metric of power for each waveform of the one or more wavelengths less sensitive to concentrations of the alcohol and determines BAC by comparing the sensitive metric of power to the less sensitive metric of power.

3. The system of claim 2 in which the sensitive metric of power and the less the sensitive metric of power include at least one of: an area under the waveform of the one or more wavelengths sensitive to concentrations of alcohol, an area under the waveform of the one or more wavelengths less sensitive to concentrations of alcohol, a square root of the area under the waveform of the one or more wavelengths sensitive to concentrations of alcohol, a square root of the area under the waveform of the one or more wavelengths less sensitive to concentrations of alcohol, a sum of the squares of the values in the waveform of the one or more wavelengths sensitive to concentrations of alcohol, a sum of the squares of the values in the waveform of the one or more wavelengths less sensitive to concentrations of alcohol, a root mean square of the values in the waveform of the one or more wavelengths sensitive to concentrations of alcohol, a root mean square of the values in the waveform of the one or more wavelengths sensitive to concentrations of alcohol, a metric of peak power, a metric of average power, or a metric of weighted average power.

4. The system of claim 1 in which the processing subsystem is configured to:
perform frequency domain analysis of each waveform of the one or more wavelengths sensitive to concentrations of alcohol and produces a sensitive power spectral density curve corresponding to hemodynamic oscillations over a predetermined frequency range,
perform frequency domain analysis of each waveform of the one or more wavelengths less sensitive to concentrations of alcohol and produces a less sensitive power spectral density curve corresponding to hemodynamic oscillations over the predetermined frequency range,
calculate a metric of power in the sensitive power spectral density curve,
calculate a metric of power in the less sensitive power spectral density curve, and
determine the BAC by comparing the metric of power in the sensitive power spectral density curve to the metric of power in the less sensitive power spectral density curve.

5. The system of claim 4 in which the processing subsystem is configured to determine BAC by calculating an area under the sensitive power spectral density curve, calculating an area under the less sensitive power spectral density curve, and determining a ratio of the calculated area under the sensitive power spectral density curve to the calculated area under the area under the less sensitive power spectral density curve.

6. The system of claim 4 in which the predetermined frequency range is in the range of about 0.5 to about 3 Hz.

7. The system of claim 1 in which the one or more wavelengths sensitive to concentrations of alcohol are in the range of about 1500 nm to about 2400 nm.

8. The system of claim 1 in which the one or more wavelengths less sensitive to concentrations of alcohol are in the range of about 1,200 nm to about 1,900 nm.

9. The system of claim 1 in which the processing subsystem is configured to rapidly and sequentially turn on and turn off one or more of the one or more emitters emitting light at the one or more wavelengths sensitive to concentrations of alcohol and emitting light the one or more wavelengths less sensitive to concentrations of alcohol in the blood of the human subject to multiplex the signal to the one or more detectors.

10. The system of claim 1 in which the one or more emitters and the one or more detectors are adapted to be placed against the skin of a portion of a human subject adjacent to each other.

11. The system of claim 1 in which the one or more emitters are adapted to be placed against the skin on a portion of a human subject and the one or more detectors are adapted to be placed against the skin of a human subject on the opposite side of the portion of the human subject.

12. A noninvasive blood alcohol concentration (BAC) measurement method, the method comprising:
emitting light at one or more wavelengths sensitive to concentrations of alcohol in blood of a human subject;
emitting light at one or more wavelengths less sensitive to concentrations of alcohol in the blood the human subject;
detecting the light emitted at the one or more wavelengths sensitive to concentrations of alcohol;
detecting the light emitted at one or more wavelengths less sensitive to concentrations of alcohol;
generating a time-varying waveform for each of the one or more wavelengths sensitive to concentrations of alcohol and a time-varying waveform for each of one or more wavelengths less sensitive to concentrations of the alcohol; and
comparing at least one waveform for each of the one or more wavelengths sensitive to concentrations of alcohol and at least one waveform for each of the one or more wavelengths less sensitive to concentrations of alcohol to determine the BAC.

13. The method of claim 12 including determining a sensitive metric of power in each waveform of the one or more wavelengths sensitive to concentrations of alcohol and determining a less sensitive metric of power for each waveform of the one or more wavelengths less sensitive to concentrations of the alcohol.

14. The method of claim 13 including determining BAC by comparing the sensitive metric of power to the less sensitive metric of power.

15. The method of claim 13 in which the sensitive metric of power and the less the sensitive metric of power include at least one of: an area under the waveform of the one or more wavelengths sensitive to concentrations of alcohol, an area under the waveform of the one or more wavelengths less sensitive to concentrations of alcohol, a square root of the area under the waveform of the one or more wavelengths sensitive to concentrations of alcohol, a square root of the area under the waveform of the one or more wavelengths less sensitive to concentrations of alcohol, a sum of the squares of the values in the waveform of the one or more wavelengths sensitive to concentrations of alcohol, a sum of the squares of the values in the waveform of the one or more wavelengths less sensitive to concentrations of alcohol, a root mean square of the values in the waveform of the one or more wavelengths sensitive to concentrations of alcohol, a root mean square of the values in the waveform of the one or more wavelengths sensitive to concentrations of alcohol, a metric of peak power, a metric of average power, or a metric of weighted average power.

16. The method of claim 12 including performing frequency domain analysis of each time-varying waveform of the one or more wavelengths sensitive to concentrations of alcohol and producing a sensitive power spectral density curve corresponding to hemodynamic oscillations over a predetermined frequency range, performing frequency domain analysis of each waveform of the one or more wavelengths less sensitive to concentrations of alcohol and produces a less sensitive power spectral density curve corresponding to hemodynamic oscillations over the predetermined frequency range, calculating a metric of power in the sensitive power spectral density curve, calculating a metric of power in the less sensitive power spectral density curve, and determining the BAC by comparing the metric of power in the sensitive power spectral density curve to the metric of power in the less sensitive power spectral density curve.

17. The method of claim 16 in which the BAC is determined by calculating an area under the sensitive power spectral density curve, calculating an area under the less sensitive power spectral density curve, and determining a ratio of the calculated area under the sensitive power spectral density curve to the calculated area under the area under the less sensitive power spectral density curve.

18. The method of claim 16 in which the predetermined frequency range is in the range of about 0.5 to about 3 Hz.

19. The method of claim 12 in which the one or more wavelengths sensitive to concentrations of alcohol are in the range of about 1500 nm to about 2400 nm.

20. The method of claim 12 in which the one or more wavelengths less sensitive to concentrations of alcohol are in the range of about 1,200 nm to about 1,900 nm.

21. The method of claim 12 including rapidly and sequentially emitting light at the one or more wavelengths sensitive to concentrations of alcohol and rapidly and sequentially emitting light the one or more wavelengths less sensitive to concentrations of alcohol.

22. The method of claim 12 including emitting only light in one narrow frequency range sensitive to concentrations of alcohol in the blood and/or emitting only light in one narrow frequency range less sensitive to concentrations of alcohol.
